(19) United States
(12) Patent Application Publication
(10) Pub. No.: US 2023/0364404 A1
(43) Pub. Date: Nov. 16, 2023

(54) ULTRASOUND FLEXURAL WAVEGUIDE

(71) Applicant: Suono Bio, Inc., Cambridge, MA (US)

(72) Inventors: Scott Kellogg, Mattapoisett, MA (US); Carl Schoellhammer, Cambridge, MA (US); Albert Farinha, Barrington, RI (US)

(21) Appl. No.: 18/026,027

(22) PCT Filed: Sep. 13, 2021
(86) PCT No.: PCT/US2021/050063

§ 371 Date: Mar. 13, 2023

Related U.S. Application Data
(60) Provisional application No. 63/222,114, filed on Jul. 15, 2021.
(60) Provisional application No. 63/077,903, filed on Sep. 14, 2020.

(51) Int. Cl. A61M 37/00; A61B 17/00; B06B 1/06

(52) U.S. Cl. CPC A61M 37/0092; A61B 17/00234; B06B 1/0611; A61B 2017/00336; B06B 2201/76

(57) ABSTRACT

The present invention provides for devices and methods that improve in vivo delivery of a drug to a target tissue by transmitting ultrasonic energy to a liquid comprising a drug at the site of the target tissue. The ultrasonic energy improves delivery of the drug to the target tissue.

### FIELD OF THE INVENTION

The invention relates to device and methods for drug delivery using ultrasonic vibrations.

### BACKGROUND

Mucosal membranes are a major obstacle for targeted drug delivery in patients in need of drug treatment. Particulate matter, including drug compounds, get trapped by the mucous and cannot reach their targets. The presence of mucosal membranes in multiple organ systems, including the lungs, colon, vagina, and bladder, poses a challenge for many targeted drug delivery methods. This is further exacerbated in conditions, such as cystic fibrosis, in which the coating of the mucosal membrane is further occluded by mucus. Presently, agents are often delivered together with a drug to dissolve thick mucus. However, these methods are frequently ineffective and the interaction between these agents and the drug to be delivered can impact the efficacy of the drugs or cause intolerable side effects. As a result, thousands of individuals suffer from ineffective targeted drug treatments or endure generalized treatment with potentially devastating off-target effects.

### SUMMARY

The present invention provides for devices and methods that improve in vivo delivery of a drug to a target tissue by transmitting ultrasonic energy to a liquid comprising a drug at the site of the target tissue. The ultrasonic energy improves delivery of the drug to the target tissue. Devices and methods of the present invention deliver the ultrasonic energy to the liquid comprising the drug while safely preventing direct contact between the source of the ultrasonic energy and the tissue.

Aspects of the invention provide a device comprising an ultrasound transducer capable of vibrating at an ultrasonic frequency. The device further comprise a probe comprising a flexible wire that is coupled to the ultrasound transducer and a flexible sheath encapsulating the flexible wire, wherein the flexible sheath comprises one or more through-holes. The flexible sheath may comprise a plurality of through-holes. Advantageously, the flexible sheath may prevent tissue in vivo from making direct contact with the flexible wire. For example, the sheath may comprise a silicone rubber to prevent contact between the flexible wire of the probe and the tissue.

The device may further comprise an adapting mechanism coupled to the ultrasound transducer to enable translation of vibrations from the transducer to the flexible wire, causing the flexible wire to be oscillated transversely to its longitudinal axis, thereby transmitting the vibrations produced by the ultrasound transducer.

The flexible wire of the probe can be any wire capable of transmitting the ultrasonic energy from the ultrasonic transducer. For example, the flexible wire may comprise nitinol or titanium. The flexible wire may consist essentially of nitinol or titanium. The flexible wire may have a diameter of less than 1 mm. For example, the flexible wire may have a diameter of 0.1 mm, 0.2 mm, 0.3 mm, 0.4 mm, 0.5 mm, 0.6, mm, 0.7 mm, 0.8 mm, 0.9 mm, 1.0 mm, or any diameters between these diameters. The flexible wire may have a diameter greater than 1.0 mm. In an aspect of the invention, the flexible wire has a diameter of 0.635 mm. The flexible wire may have a length between about 10 cm to about 30 cm. Because the sheath protects the flexible wire, the sheath may also be between about 10 cm to about 30 cm. It is understood that the length of the flexible wire and thereby the probe can be adjusted based on the location of the drug to be delivered and the avenue for reaching the target site. For example, the length of the flexible wire, the sheath, and/or the probe may be less than 1 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 35 cm, 40 cm, 45 cm, 50 cm, 60 cm, 70 cm, 80 cm, 90 cm, 100 cm or any lengths in between these lengths. The length of the flexible wire and the probe may be greater than 100 cm. The flexible wire may extend from the sheath at least one point on the sheath, thereby forming a tip. The tip may be used to concentrate the release of ultrasonic energy at the point of the tip and may be directed to the target tissue.

It is also understood that the diameter of the sheath may be adjusted based on the location of the drug delivery and the ultrasonic energy to be applied. For example, the sheath may have a diameter of about 2 mm to about 4 mm. The diameter of the sheath may be less than 1 mm, 1 mm, 2 mm, 3 mm, 4 mm, 5 mm, 6 mm, 7 mm, 8 mm, 9 mm, 10 mm, or any diameters between these diameters. The diameter of the sheath may be greater than 10 mm.

Advantageously, the flexible wire, sheath, and/or probe may be stearable. A stearable probe is able to be mechanically controlled by the user and directed to a target location. For example, the probe may be steared through a surgical incision to reach a target tissue in vivo. The probe may alternatively be steared through the passages of the subject to reach a target tissue, for example through the trachea and bronchioles of a subject to reach target lung tissue.

The invention also provides for use of the devices of the invention and methods for improving delivery of a drug to an in vivo target tissue.

Methods of the invention comprise delivering a drug to an in vivo target tissue by introducing into a body orifice of a subject a device comprising an ultrasound transducer capable of vibrating at an ultrasonic frequency, a probe comprising a flexible wire that is coupled to the ultrasound transducer; and a flexible sheath encapsulating the flexible wire, wherein the flexible sheath comprises one or more through-holes. A liquid comprising a drug is present in the body orifice and the method further comprises vibrating the flexible wire such that energy produced by the vibrations from the flexible wire exits the through-holes and interacts with the drug to thereby deliver the drug into a target tissue within the body orifice.

In aspects of the invention, the introduced into the body orifice comprises an adapting mechanism coupled to the ultrasound transducer to enable translation of vibrations from the transducer to the flexible wire, causing the flexible wire to be oscillated transversely to its longitudinal axis, thereby transmitting the vibrations produced by the ultrasound transducer.

The flexible sheath of the device introduced into the body orifice may comprise a plurality of through-holes. Advantageously, the flexible sheath may prevent tissue in vivo from making direct contact with the flexible wire. For example, the sheath may comprise a silicone rubber to prevent contact between the flexible wire of the probe and the tissue.

The flexible wire of the device introduced into the body orifice can be any wire capable of transmitting the ultrasonic energy from the ultrasonic transducer. For example, the flexible wire may comprise nitinol or titanium. The flexible wire may consist essentially of nitinol or titanium. The flexible wire may have a diameter of less than 1 mm. For example, the flexible wire may have a diameter of 0.1 mm, 0.2 mm, 0.3 mm, 0.4 mm, 0.5 mm, 0.6, mm, 0.7 mm, 0.8 mm, 0.9 mm, 1.0 mm, or any diameters between these diameters. The flexible wire may have a diameter greater than 1.0 mm. In an aspect of the invention, the flexible wire has a diameter of 0.635 mm. The flexible wire may have a length between about 10 cm to about 30 cm. Because the sheath protects the flexible wire, the sheath may also be between about 10 cm to about 30 cm. It is understood that the length of the flexible wire and thereby the probe can be adjusted based on the location of the drug to be delivered and the avenue for reaching the target site. For example, the length of the flexible wire, the sheath, and/or the probe may be less than 1 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 35 cm, 40 cm, 45 cm, 50 cm, 60 cm, 70 cm, 80 cm, 90 cm, 100 cm or any lengths in between these lengths. The length of the flexible wire and the probe may be greater than 100 cm.

It is also understood that the diameter of the sheath may be adjusted based on the location of the drug delivery and the ultrasonic energy to be applied. For example, the sheath may have a diameter of about 2 mm to about 4 mm. The diameter of the sheath may be less than 1 mm, 1 mm, 2 mm, 3 mm, 4 mm, 5 mm, 6 mm, 7 mm, 8 mm, 9 mm, 10 mm, or any diameters between these diameters. The diameter of the sheath may be greater than 10 mm.

### BRIEF DESCRIPTION OF THE DRAWINGS

FIG. 1 depicts an exemplary device according to an embodiment of the invention.

FIG. 2 depicts an exemplary device according to an embodiment of the invention.

FIG. 3 depicts a device used with an endoscope according to an embodiment of the invention.

### DETAILED DESCRIPTION

The present invention provides for devices and methods that improve in vivo delivery of a drug to a target tissue by transmitting ultrasonic energy to a liquid comprising a drug at the site of the target tissue. The ultrasonic energy improves delivery of the drug to the target tissue. In certain devices of the invention, the transducer remains external to the subject while in use, and the sheath and wire are inserted into the subject via the tube of an endoscope. Such devices are advantageous over prior ultrasound devices because they obviate the need to use a miniature transducer that fits into the endoscope tube, deliver greater ultrasound energy to target tissue, and eliminate the need for distal electrical insulation.

Ultrasonic energy has previously been considered for a number of medical uses, including diagnostic imaging, tissue ablation, and fragmentation of plaque and thrombosis. Typically, the energy produced by an ultrasonic probe is in the form of very intense, high frequency sound vibrations, resulting in fragmentation of tissue either as a result of mechanical action or “cavitation”, in which high energy ultrasound frequency applied to liquids generates vapor-filled microbubbles or “cavities”, with the rapid expansion and collapse of the cavities accompanying intense localized hydraulic shock that causes fragmentation or dissolution of the tissue. Ultrasonic devices are described, for example, in U.S. Pub. No. 2003-0176791, the contents of which are incorporated herein in their entirety.

Without being bound to a single mechanism of action, low-frequency ultrasonic energy has recently been suggested for use in influence cell function and drug delivery. However, until the present invention, the use of ultrasound to improve drug delivery in vivo has been limited. See Tharkar et al. (2019) “Nano-Enhanced Drug Delivery and Therapeutic Ultrasound for Cancer Treatment and Beyond”, Frontier in Bioengineering and Biotechnology vol. 7, art. 324; and Pitt et al. (2004) “Ultrasonic Drug Delivery—A General Review”, Expert Opin Drug Deliv. 1(1):37-56, the contents of each of which are incorporated herein in their entirety. The ultrasonic energy of the present invention may disrupt mucosal structures, allowing the drug to enter the tissues for local drug delivery. The ultrasonic energy may interact with the drug molecules to improve delivery of the drug. Additionally, by the present invention, devices are also provided for the first time that can safely apply ultrasonic energy in vivo for targeted drug delivery.

Aspects of the invention provide a device comprising an ultrasound transducer capable of vibrating at an ultrasonic frequency. The device further comprise a probe comprising a flexible wire that is coupled to the ultrasound transducer and a flexible sheath encapsulating the flexible wire, wherein the flexible sheath comprises one or more through-holes. The flexible sheath may comprise a plurality of through-holes. The through-holes may be positioned transversely along the length of the sheath. The through-holes may be positioned at locations of maximum wire vibration in order to elicit excitation and delivery of drug at multiple locations along the GI tract.

Without being bound to a single mechanism of action, the device may be configured to produce from the vibrations at least one of acoustic cavitation, micro-jetting, or micro-streaming in a liquid comprising a drug. “Acoustic cavitation” or “cavitation” means shock waves produced by ultrasonic vibration, wherein the vibration creates a plurality of microscopic bubbles which rapidly collapse, resulting in molecular collision by water molecules which collide with force, thereby producing the shock waves. “Micro-jets” are powerful streams of liquid caused by asymmetric implosion of microbubbles, for example microbubbles formed from cavitation. “Microstreaming” means the streaming flow of fluid around an oscillating object such as a gas bubble. The fluid flow is generated from the vorticity caused by the oscillation of the boundary layer surrounding, for example, an oscillating cavitation bubble. In aspects of the invention, the energy released from the device is released longitudinal to the probe. In aspects of the invention, vibrations produced by the ultrasound transducer may be transmitted along the flexible wire by flexural displacement. For example, the flexural displacement of the flexible wire may be about 30 μm to about 100 μm. It is understood that flexural displacement of the flexible wire may be adjusted to adjust the vibrations transmitted and produced by the distal end of the probe.

In aspect of the invention, the ultrasonic energy delivered by the device may be adjusted by any known methods. For example, the power transmitted to the ultrasound transducer, or the setting of the ultrasound transducer may be used to adjust the ultrasonic energy delivered. The ultrasound transducer may be tuned to adjust the frequency of the ultrasonic transducer. “Tuning” is a process of adjusting the frequency of the ultrasonic generator means to select a frequency that establishes a standing wave along the length of the probe. The sheath of the probe may also substantially prevent transmission of energy generated by the ultrasound transducer and transmitted to the probe from being provided to the surrounding environment. The flexible sheath may comprise one or more through-holes that allow for the controlled or targeted release of energy generated by the device to the surrounding energy. Additionally, the sheath of the device may prevent mucosal tissue in vivo from making direct contact with the flexible wire. For example, the sheath may comprise a silicone rubber to prevent contact between the flexible wire of the probe and the tissue. A “probe” broadly means to a device capable of being adapted to an ultrasonic generator means, which is capable of propagating the energy emitted by the ultrasonic generator means along its length, and is capable of acoustic impedance causing transformation of ultrasonic energy into mechanical energy. A “sheath” means to an apparatus for covering, encasing, or shielding in whole or in part, a probe or port thereof connected to an ultrasonic generation means.

The invention can be used deliver a broad range of therapeutic agents. For example, without limitation, the drug may be a small molecule, a large molecule, a synthetic molecule or semi-synthetic molecule, a protein, a peptide, a peptidic molecule, a glycoprotein, a nucleoside, a nucleotide, an antibody or a antibody fragment, for example a monoclonal humanized or non-humanized antibody, a gene editing agent, or an agent for effecting RNA interferences, for example dsRNA, miRNA, siRNA, antisense RNA, or antisense oligonucleotides. The drug may be delivered in any composition for delivery of the drug, for example in a composition together with any standard carriers, including saline, buffers, water, and emulsions, such as water/oil emulsions, stabilizers, and preservatives.

In embodiments of the invention, ultrasound energy delivered from the device may remove debris from the mucosal walls of the GI tract or “clean” them to allow better drug delivery via diffusion or cavitation. The ultrasound energy may also open pores in targeted cells, and the pores may facilitate entry of a therapeutic agent into the cells.

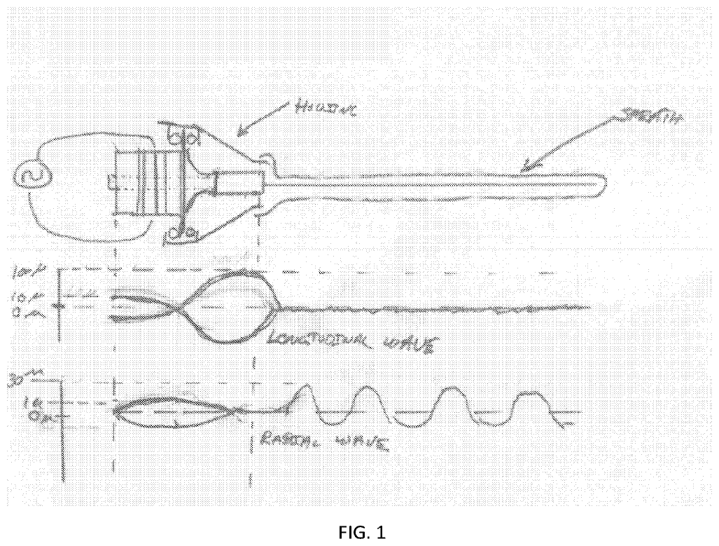

FIG. 1 discloses an exemplary device according to an embodiment of the invention. An ultrasonic transducer, for example a bolted Langevin ultrasound transducer as described below, is mechanically coupled to a flexible wire that is encapsulated in flexible protective and perforated sheath (the probe). The flexible wire may be loosely encapsulated by the flexible wire. When the sheath is inserted endoscopically in vivo and ultrasonically activated in drug containing surrounding fluid, the flexing wire produces acoustic cavitation, micro-jetting, and/or micro-streaming that enhances the trans-mucosal transport of the drug product.

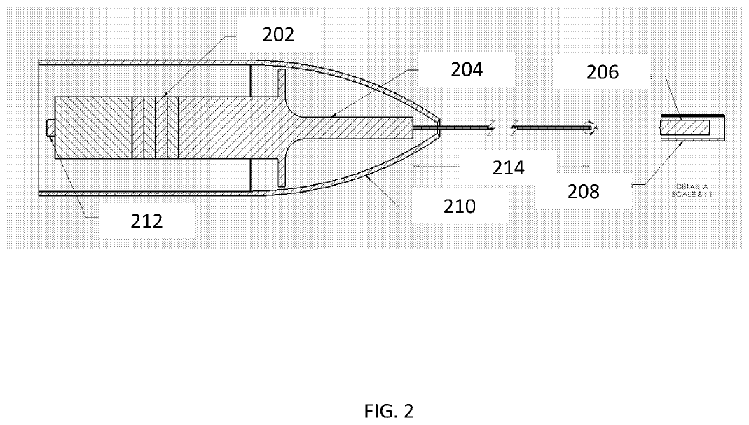

FIG. 2 shows an exemplary device according to an embodiment of the invention. The device includes a flexible wire 206 disposed at least partially within a flexible sheath 208. The device may include a house 210 that is configured to fit at least partially into an endoscope. The device may include a transducer 204 that includes piezo ceramics 202. The transducer 210 remains external to the body of the subject while the device is in use. The device may have a bolt 214 at a proximal end of the transducer.

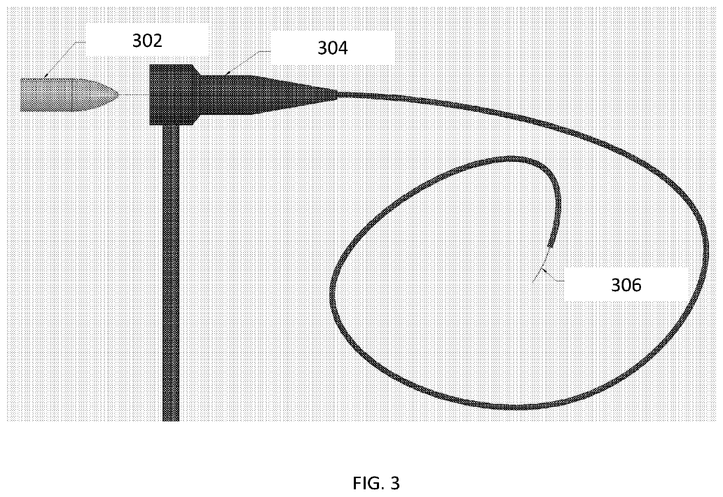

FIG. 3 shows a device used with an endoscope according to an embodiment of the invention. The transducer and housing 302 remain external to the subject and are configured to fit into the proximal end of an endoscope 304. The flexible wire and sheath 306 are attached at their proximal ends to the transducer and housing 302 and extend into the proximal end of the endoscope, through the insertion tube, and beyond the distal tip of the insertion tube.

It is understood that an ultrasonic transducer converts electrical energy to mechanical energy. Accordingly, any compatible electrical source can be used with the present invention. For example, a medical-grade (Type B, BF, or CF) electrical generator produces a substantially sinusoidal waveform at a frequency matched and phase-locked to the resonate frequency of the ultrasound transducer. In preferred aspects of the invention, the output is 50 kHz, 10 to 100 Vrms, and 100 to 1000 mA with output wattage ranging from about 10 W to about 100 W. The amount of energy to be applied to a particular site is a function of the amplitude and frequency of vibration of the probe, as well as the longitudinal length of the probe tip, the proximity of the tip to a tissue, and the degree to which the probe tip is exposed to the tissues. Control over this last variable can be effectuated through the sheath of the present invention. The use of a sheath further diminishes or prevents the local temperature rise. Accordingly, the sheath of the present invention may provide a means of insulating surrounding tissue from the thermal side effects of the ultrasonic probe. The length and diameter of the sheath used in a particular device or method is dependent on the type of probe used, the degree to which the probe length will be inserted into the patient, and the degree of shielding that is required based on the specific areas to be targeted.

For example, in an application whereby prostate tissue or the bladder is targeted via an intra-urethral route with the ultrasonic probe of the present invention, the sheath must be of a sufficient length to protect the tissue of the urethra, of a sufficient outside diameter to facilitate insertion of the sheath into the urethra, and a sufficient inside diameter capable of accepting the probe. The exact dimensions of the sheath, including its length and diameter, are determined by requirements of a specific medical procedure. The position and size of the sheath aperture, or number and positions of the fenestrations/perforations, or the presence of a bevel on the sheath terminus to provide a means for tissue manipulations, will likewise be determined by the type of procedure, and the requirements of the particular patient.

In one aspect of the invention, the sheath comprises an inner sheath and an outer sheath. The outer sheath may be connected to a retraction trigger, by one or more articulation means, such as wires, which is capable of moving the outer sheath with respect to the inner sheath. Thus, a sheath may be retractable. Each wire comprises a first end and a second end. The first end is affixed to the outer sheath, while the second end is affixed to a retraction trigger. When the outer sheath is slid back away from the terminus of the inner sheath the tissues are exposed to cavitation energy emitted by the probe. A sheath may also be retractable in relation to the wire so that retraction of the sheath allows the distal end of the wire to protrude from the sheath.

In another aspect, the sheath is flexible. Articulation wires comprising two ends, are connected to the sheath and an articulation handle. When the articulation handle is manipulated, for example, pulled axially inward, the flexible sheath will bend or articulate in a bending or articulation direction, thereby causing the ultrasonic probe to bend or articulate in articulation direction. In this way, the ultrasonic probe can be used to reach locations that are not axially aligned with the lumen or vessel through which the sheath and probe are inserted.

The electrical output is connected to the ultrasound transducer that converts the electrical signal into mechanical vibration. A bolted Langevin transducer consists of a steel central retaining bolt that sandwiches 4 piezo ceramic elements between a steel end-bell and a titanium (6Al4V) fore-bell/acoustic horn. The bolts compress the piezo stack assembly at approximately 1,000 to 4,000 psi and the horn amplifies the longitudinal vibration. A preferred transducer is approximately 1.5 inches (38 mm) in length, sandwiching 0.750 inch (19 mm) diameter piezo (type PZT-8) elements. The preferred transducer resonates at approximately 50 kHz. The longitudinal displacement at the distal tip of the transducer ranges from 10 to 100 The center of the transducer may incorporate a mounting feature that secures the transducer to an outer housing. This central mounting feature may reside at a longitudinal node in the overall waveguide where is the longitudinal displacement is essentially zero making it suitable for mounting. The node is the region of maximum energy emitted by an ultrasonic probe on or proximal to a position along the probe. As there is radial displacement at the longitudinal node, mounting may be further acoustically isolated from the housing using dampening silicone o-rings. The outer housing may be advantageous because it allows for incorporation of a hand-piece that prevents contact by the user with the transducer and provides for a point of connection to a protective endoscopic probe.

The distal tip of the fore-bell/acoustic horn is mechanically coupled to the proximal end of the flexible wire. When activated the longitudinal displacement produced by the transducer produces sympathetic flexural vibration in the wire. The flexural vibration is function of modulus of elasticity and diameter of the wire. In preferred aspect of the invention the wire may be nitinol or titanium (NiTi/6Al4V). The wire and/or sheath may have a size suitable to fit into the working channel of an endoscope. The wire may have a diameter of about 0.1 mm, about 0.2 mm, about 0.3 mm, about 0.4 mm, about 0.5 mm, about 0.6 mm, 0.025 inches (0.635 mm), about 0.8 mm, about 1.2 mm, about 1.5 mm, about 2.0 mm, or about 2.5 mm. The wire may be 10 to 30 cm in length as dedicated by the requirements of the specific endoscopic indication. For applications that involve insertion of the sheath and wire through the rectum, the wire may be at least 50 cm, at least 60 cm, at least 70 cm, at least 80 cm, at least 90 cm, at least 100 cm, at least 120 cm, at least 150 cm, about 50 cm, about 60 cm, about 70 cm, about 80 cm, about 90 cm, about 100 cm, about 120 cm, about 130 cm, about 140 cm, about 150 cm, about 160 cm, about 170 cm, about 180 cm, or about 200 cm. In a preferred aspect the flexural displacement of the wire is 30 to 100 μm and suitable for inducing acoustic cavitation, micro-jetting, and/or micro-streaming in the surrounding fluid.

The flexural wire may be loosely encapsulated in a protective flexible endoscopic outer sheath. The sheath is perforated to provide for acoustic cavitation, micro-jetting, and/or micro-streaming in the drug containing surrounding fluid while protecting the surrounding mucosal tissue from making direct contact with the wire. The sheath may be 2 to 4 mm in diameter and 10 to 30 cm in length. The sheath may be made of a soft elastomer, such as medical grade (biocompatible/temporary contact) silicone rubber or pebax.

The sheath may have an opening or hole at its distal end that allows a therapeutic agent contained in the sheath to be irrigated or injected to a target site or tissue. The sheath may have multiple lumens that extend along its length and are physically separated from each other to allow for simultaneous delivery of multiple different therapeutic compositions, such as drugs or irrigation fluids. The sheath may also have at least two longitudinal holes along its length in order to separate the drug from the ultrasonically-vibrating wire until a preferred location when the drug becomes excited with the wire. Additional longitudinal holes could allow for multiple yet separate drug delivery. The sheath may contain balloon sections distal and proximal to the distal wire tip and transverse through-holes in order to limit delivery and ultrasound treatment of drug to isolated sections of the GI tract.

In a preferred aspect of the invention the endoscopic inserted sheath and containing wire (the probe) are single use and disposable and incorporate a feature to attach and detach it from a reusable hand-piece housing that comprises the ultrasonic transducer.

In aspects of the invention, the device of the present invention may further comprise an irrigation channel. The sheath may be adapted to an irrigation means, for example, a peristaltic pump or other such device for delivering liquids, including therapeutic substances, under controlled flow rates and pressures, and the sheath directs fluid to the location of the probe. The irrigation channel can be manufactured out of the same material as the sheath provided it is of a sufficient rigidity to maintain its structural integrity under the positive pressure produced by the flow of fluid produced by the irrigation means. Such an irrigation channel is provided either inside the lumen of the sheath, or along the exterior surface of the sheath, or both. The irrigation channel can be a second hollow sheath nested within the first sheath, or the irrigation channel can be formed in the body of the sheath. The probe itself may have one or more grooves defining irrigation channels, and fluid is directed along the probe length between the interior surface of the sheath and the exterior surface of the probe, as directed by the irrigation channels. Irrigation fluids may provide a means of cooling the probe. Advantageously, the irrigation channels may deliver the liquid comprising the drug for which the trans-mucosal delivery of which is improved by the energy provided by the probe. The sheath itself, or an irrigation sheath contained within the first sheath can provide a means of introducing the drug or pharmaceutical formulation to the site of probe activity. The ultrasonic energy further provides a means for assisting the drug in penetrating the mucosal membrane.

The devices of the present invention may further comprise both an irrigation and an aspiration channel. As described above with regard to the irrigation channel, the channels may be located within the sheath lumen, or exterior to the sheath, or a combination of the two, and can be proximal or distal to the other channel provided they are not in direct communication. Likewise, the probe itself has a plurality of grooves defining aspiration channels and irrigation channels, and fluid is directed along the probe length between the interior surfaces of the sheaths and the exterior surface of the probe, as directed by the aspiration and irrigation channels. In aspect of the invention, the sheath comprises a means for directing, controlling, regulating, and focusing the energy emitted by the probe, an aspiration means, an irrigation means, or any combination of the above.

The device may allow for the manipulation of tissues, comprising a surface that is capable of manipulating tissues near the distal end of the probe. In this aspect, the terminus of the sheath may be closed, such that the sheath insulates tissues from the energy emitted by the probe and can be used to push tissues away from the distal end. Alternatively, the sheath may comprise a beveled or arcutate surface at the distal end, capable of providing a means for hooking, grasping, or otherwise holding a tissue in proximity to the probe. The sheath may allow for the introduction of another surgical device, for example, flexible biopsy forceps, capable of manipulating tissues into a tissue space, such that the surgical device can hold the tissue in proximity with the probe.

In a further aspect, the internal surface of the sheath provides a means to amplify or focus energy from the probe. In this aspect, the interior surface of the sheath comprises at least one structure or reflective element, that extends into the sheath lumen. The reflective element may be planar, or arcutate, or a combination of these shapes. Reflective elements of the present invention may be fabricated from the same material as the sheath, or may use different materials that optimize the reflective properties of the elements. Since the energy reaches a maximum at nodes along the probe, the interval of the nodes being determined by the ultrasonic frequency at which the generator operates, the spacing of the reflective elements in the sheath is determined by the intended operating frequency of the ultrasonic device. Similarly, the number of nodes along the probe, is determined by the length of the probe and the frequency. As such, the number of reflective elements is determined by the length of the probe and the operating frequency. For example, an ultrasonic device operating at a frequency of approximately 25 kHz employing a probe with a length at the thinnest interval 22 of about 3 centimeters, will display about seven nodes approximately 2 millimeters wide, spaced about 2 millimeters apart. Energy will radiate circumferentially around the probe at these nodes. A sheath useful with such a probe would comprise, for example but not limited to, a cylindrical sheath about at least 3 centimeters in length further comprising seven reflective elements, approximately 2 millimeters wide, spaced about 2 millimeters apart, positioned with respect to the probe such that the reflective elements, are centered over the nodes. Since the energy emitted by the probe radiates circumferentially from a node, the reflective elements can extend radially from the interior wall of the sheath into the sheath lumen, for example, degrees around the interior of the sheath, while the remaining 90 degrees has no reflective element and thereby provides a means for channeling the cavitation energy from the node to a position distal to the node. The channeling means of the present example may be a region where no reflective element is present, or where the shape or angle is altered compared to the reflective element, or any other such means of directing energy from the area of the node to a position distal to the node.

The device of the present invention may comprise a means of viewing the site of probe action, that is the point of contact between the liquid comprising the drug and the distal end of the probe. This may include an illumination means and a viewing means. In one aspect, the sheath of the present invention comprises a means for containing or introducing (if external to the sheath) an endoscope, or similar optical imaging means. In another aspect, the ultrasound medical device is used in conjunction with an imaging system, for example, MRI, or ultrasound imaging-in particular color ultrasound. The action of the probe may echogenically produce a pronounced and bright image on the display. The sheath in this embodiment shields the probe, thereby reducing the intensity of the probe image and enhancing the resolution of the image by decreasing the contrast between the vibrating probe and the surrounding tissues.

Sheath materials useful for the present invention include any material with acoustical or vibrational dampening properties capable of absorbing, containing, or dissipating the cavitation energy emitted by the probe tip. Preferably, such materials must be capable of being sterilized by, for example, gamma irradiation or ethylene oxide gas (ETO), without losing their structural integrity. Such materials include but are not limited to, plastics such as polytetrafluoroethylene (PTFE), polyethylene, polypropylene, silicone, polyetherimide, or other such plastics that are used in medical procedures. Ceramic materials can also be used, and have the added benefit that they may be sterilized by autoclaving. Combinations of the aforementioned materials can be used depending on the procedure. Alternatively, as described above, the sheath may comprise a material that is disposable for single-use probes.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A device comprising:
an ultrasound transducer capable of vibrating at an ultrasonic frequency;
a probe comprising a flexible wire that is coupled to the ultrasound transducer; and
a flexible sheath encapsulating the flexible wire, wherein the flexible sheath comprises one or more through-holes.

2. The device of claim 1, further comprising an adapting mechanism coupled to the ultrasound transducer to enable translation of vibrations from the transducer to the flexible wire, causing the flexible wire to be oscillated transversely to its longitudinal axis, thereby transmitting the vibrations produced by the ultrasound transducer.

3. The device of claim 1, wherein the flexible sheath comprises a plurality of through-holes.

4. The device of claim 1, wherein the flexible sheath prevents mucosal tissue in vivo from making direct contact with the flexible wire.

5. The device of claim 1, wherein the flexible wire comprises nitinol or titanium.

6. The device of claim 1, wherein the flexible wire has a diameter of 0.635 mm.

7. The device of claim 1, wherein the flexible wire has a length of about 10 cm to about 30 cm.

8. The device of claim 1, wherein the flexible sheath has a diameter of about 2 mm to about 4 mm.

9. The device of claim 1, wherein the flexible sheath has a length of about 10 cm to about 30 cm.

10. The device of claim 1, wherein the flexible sheath comprises silicone rubber.

11. A method for delivering a drug to an in vivo target tissue, the method comprising:
introducing into a body orifice of a subject a device comprising an ultrasound transducer capable of vibrating at an ultrasonic frequency, a probe comprising a flexible wire that is coupled to the ultrasound transducer; and a flexible sheath encapsulating the flexible wire, wherein the flexible sheath comprises one or more through-holes; wherein a liquid comprising a drug is present in the body orifice; and
vibrating the flexible wire such that energy produced by the vibrations from the flexible wire exits the through-holes and interacts with the drug to thereby deliver the drug into a target tissue within the body orifice.

12. The method of claim 11, further comprising an adapting mechanism coupled to the ultrasound transducer to enable translation of vibrations from the transducer to the flexible wire, causing the flexible wire to be oscillated transversely to its longitudinal axis, thereby transmitting the vibrations produced by the ultrasound transducer.

13. The method of claim 11, wherein the flexible sheath comprises a plurality of through-holes.

14. The method of claim 11, wherein the flexible sheath prevents mucosal tissue in vivo from making direct contact with the flexible wire.

15. The method of claim 11, wherein the flexible wire comprises nitinol or titanium.

16. The method of claim 11, wherein the flexible wire has a diameter of 0.635 mm.

17. The method of claim 11, wherein the flexible wire has a length of about 10 cm to about 30 cm.

18. The method of claim 11, wherein the flexible sheath has a diameter of about 2 mm to about 4 mm.

19. The method of claim 11, wherein the flexible sheath has a length of about 10 cm to about 30 cm.

20. The method of claim 11, wherein the flexible sheath comprises silicone rubber.

21. The device of claim 1, wherein the flexible wire is at least 100 cm in length.

22. The device of claim 21, wherein the flexible wire is about 150 cm in length.

23. The device of claim 1, wherein the flexible wire is about 1 mm in diameter.

24. The device of claim 1, wherein the flexible sheath is configured to fit within an endoscope.

25. The device of claim 1, wherein the flexible sheath comprises a plurality of lumens that extend along a length of the sheath and are physically separated from each other.

26. The device of claim 25, wherein each of the plurality of lumens comprises a different therapeutic composition.

27. The device of claim 1, wherein the flexible sheath comprises a hole at its distal end.

28. The device of claim 27, wherein the flexible sheath is retractable and wherein a distal portion of the flexible wire extends through the hole at the distal end of the flexible sheath the flexible sheath is retracted.

29. The device of claim 1, wherein the sheath comprises at least two longitudinal holes.

30. The device of claim 1, wherein the sheath comprises at least one of a balloon section proximal to a distal tip of the flexible wire and a balloon section distal to the distal tip of the wire, each balloon section serving as a barrier to longitudinal diffusion of contents within the flexible sheath.
